(19) United States
(12) Patent Application Publication
(10) Pub. No.: US 2021/0295967 A1
(43) Pub. Date: Sep. 23, 2021

(54) INDIVIDUAL HEALTH INFORMATION SYSTEM

(71) Applicant: KABUSIKIGAISYA DIGIMED, Wakayama (JP)

(72) Inventor: Yoshikatsu Nakanishi, Wakayama (JP)

(21) Appl. No.: 17/059,192

(22) PCT Filed: Aug. 30, 2019
(86) PCT No.: PCT/JP2019/034063

§ 371 Date: Nov. 25, 2020

(30) Foreign Application Priority Data
Dec. 14, 2018 (JP) 2018-234480
Aug. 30, 2019 (JP) PCT/JP2019/034063

(51) Int. Cl. G16H 10/65; G16H 40/67; G16H 80/00

(52) U.S. Cl. CPC G16H 10/65; G16H 80/00; G16H 40/67

(57) ABSTRACT

In an individual health information system of the present invention, health information such as a medical record and medication information of a patient is recorded in an information communication terminal constituted by a smartphone in advance, and a dedicated application is activated. After the patient is transported in an ambulance, when a medical professional such as a paramedic causes the dedicated application to operate by calling with a voice or applying an activation signal from an information processing device through Bluetooth (registered trademark), the health information is mirrored to the information processing device through Bluetooth (registered trademark), and necessary information can be retrieved by a remote operation as appropriate. With this, even if the name of the patient is unknown, or health information cannot be obtained from his or her family doctor's hospital due to the mobile phone channel being interrupted, the health information can be acquired directly, reliably, and stably, and as a result, treatment can be performed quickly and appropriately.

### TECHNICAL FIELD

The present invention relates to a system that, as a result of recording, in a mobile communication terminal such as a smartphone, health information such as hospital visiting histories and medication histories of individuals, namely an owner thereof and preferably members relating to the owner such as family members, is useful when medical professionals such as paramedics and doctors perform treatment when a member is in poor physical condition such as a case where emergency transportation is needed.

### BACKGROUND ART

As a result of recording the health information, specifically the health information of individuals such as examination (diagnostics) results (medical records), test results (biochemical tests, images, and the like), and medication histories (prescriptions, medication notebooks), in a mobile communication terminal, the information can be utilized, when the owner thereof visits a hospital as an outpatient, for preventing duplication of medicine or the like, by the information being shown to the doctor, for example. Also, in the case where the accurate disease name is difficult to be determined such as a case of so-called intractable diseases as well, by referring to the past health information, a doctor can determine the accurate disease name or the member can use it when seeking a second opinion, and this is extremely effective.

In view of this, the inventor of the present invention has previously proposed a system in which such pieces of health information of individuals are aggregated, in Patent Document 1. In the conventional technology, while establishing an aggregation facility for aggregating the health information on a medical professional (medical institutions) side, a unique identification code (ID) is assigned to each examinee (owner). Also, when medical institutions perform medical practices such as examination and medication, the obtained pieces of health information are aggregated in association with the identification code, and as a result, the health information can be shared between medical institutions. The obtained health information can also be downloaded to a smartphone or a tablet terminal of the owner (examinee) so as to be able to be carried along.

### CITATION LIST

### Patent Document

Patent Document 1: JP 6059303B

### DISCLOSURE OF INVENTION

### Problem to be Solved by the Invention

With the abovementioned conventional technology, the aggregated health information of individuals can be stored in mobile communication terminals, and can be shown to doctors or the like as appropriate. However, in a situation in which an examinee (owner) cannot operate the mobile communication terminal, e.g., in a situation in which the examinee is being transported in an emergency, there are cases where valuable health information cannot be confirmed due to being hampered by a password lock of the mobile communication terminal. Also, even if an attempt is made to download the health information of the individual through the aggregation center, there are cases where downloading is not possible because the identification code (ID) unique to the examinee (owner) is not known (it is not possible to read the identification information from the mobile communication terminal), or the wireless wide area network is interrupted (becomes unstable) due to the ambulance traveling underground or in a tunnel.

An object of the present invention is to provide an individual health information system in which health information can be reliably taken out from mobile communication terminals in which health information of individuals is recorded, in an emergency situation as well.

### Means for Solving Problem

In order to realize the abovementioned object, an individual health information system of the present invention is for displaying health information that is in a mobile communication terminal such as a smartphone that records the health information on an information processing device such as a smartphone or a tablet terminal that a medical professional possesses, and the mobile communication terminal causes the health information to be mirrored to the information processing device by short range wireless communication (locally) in response to a predetermined activation sign from a medical professional side.

Therefore, even if the owner's name is unknown, or even in a case where the health information of the owner cannot be obtained from his or her family doctor's hospital on the medical professional side such as an ambulance or an ambulance helicopter due to a large-scale wireless communication network such as a mobile phone channel being unstable, the health information can be acquired directly, reliably, and stably, and the medical professional can perform treatment quickly and appropriately.

### BRIEF DESCRIPTION OF DRAWINGS

FIG. 1 is a diagram illustrating a configuration of an individual health information system according to one embodiment of the present invention.

FIG. 2 is a block diagram illustrating electrical configurations of a mobile communication terminal on an owner (examinee, patient) side and an information processing device on a medical professional side that constitute the individual health information system.

FIG. 3 is a block diagram illustrating the overall configuration of the individual health information system, and shows a state in which an examinee (patient) is in under normal conditions.

FIG. 4 is a block diagram illustrating the overall configuration of the individual health information system, and shows a state in which the examinee is transported in an emergency situation.

FIG. 5 is a diagram illustrating one example of a top screen that is displayed in a mobile communication terminal, and is for selecting desired health information, in the individual health information system.

FIG. 6 is a diagram illustrating one example of a setting screen that is a result of input from FIG. 5.

FIG. 7 is a diagram illustrating one example of an input screen for accessing health information of an examinee (patient) in the individual health information system.

FIG. 8 is a diagram illustrating one example of a display screen of detailed individual health information received from an aggregation server in the individual health information system.

### DESCRIPTION OF THE INVENTION

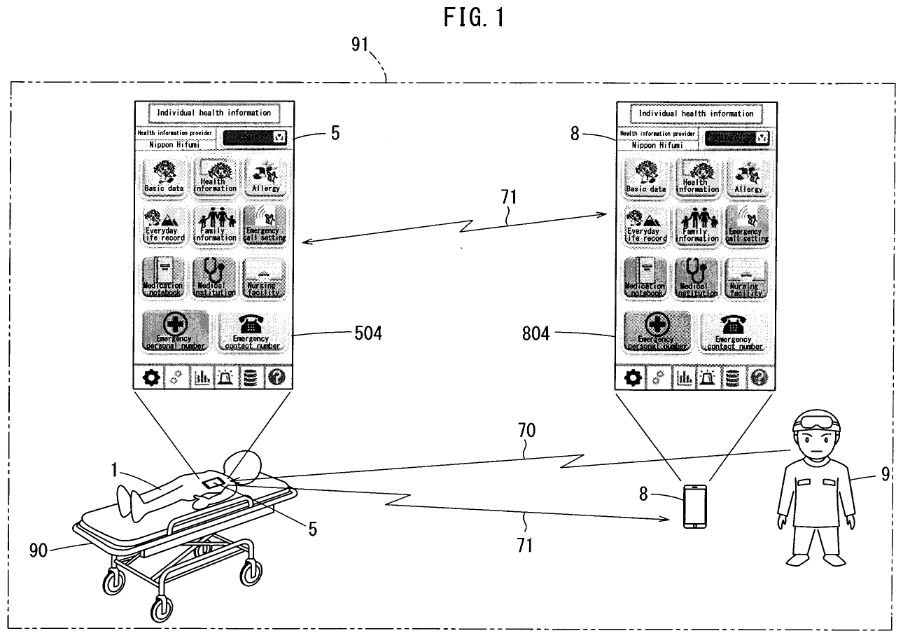

FIG. 1 is a diagram illustrating a configuration of an individual health information system according to one embodiment of the present invention. First, as a minimum configuration, the individual health information system of the present embodiment includes a mobile communication terminal 5 and an information processing device 8, as shown in FIG. 1. The mobile communication terminal 5 is a terminal having a communication function, and can record pieces of health information of members including at least an owner 1 of the terminal and family members related to the owner 1. The information processing device 8 is possessed by a medical professional 9, and the medical professional 9 performs treatment when the member is in poor physical condition. In the example in FIG. 1, a situation is shown in which the owner 1 is in poor physical condition, is placed on a stretcher 90, and is conveyed while being accommodated in an ambulance 91, which serves as an accommodation place, and the medical professional 9 that performs a treatment on the owner 1 is a paramedic.

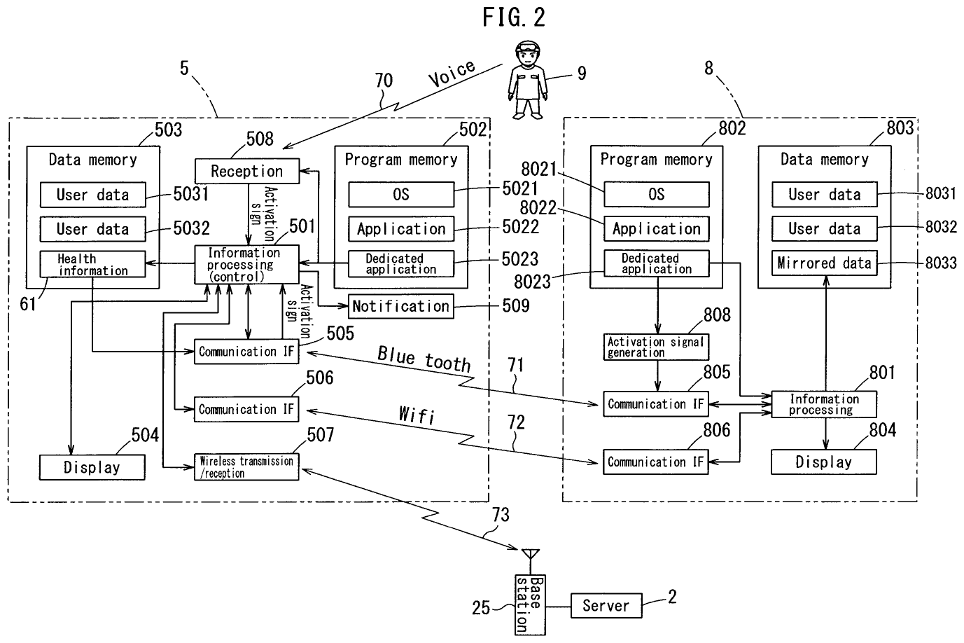

FIG. 2 is a block diagram illustrating electrical configurations of the mobile communication terminal 5 and the information processing device 8. The mobile communication terminal 5 includes an information processing unit 501 constituted by a central information processing device and the like, a program memory 502, a data memory 503, a display unit 504, a communication interface 505 of Bluetooth (registered trademark) 71, a communication interface 506 of Wi-Fi (registered trademark) 72, and a wireless transceiver 507 of a mobile phone channel 73. The mobile communication terminal 5 is constituted by a smartphone, and includes standard functions that are mounted on a smartphone such as a camera, a flash, a touch panel sensor, and an antenna, in addition to the above-described constituent elements.

The information processing unit 501 serving as control means functions based on an OS 5021 and an application program 5022 that are stored in the program memory 502, and can display data 5031 or 5032 stored in the data memory 503 in the display unit 504, and exchange data using the communication interfaces 505 and 506 and the wireless transceiver 507.

The information processing device 8 also includes an information processing unit 801 constituted by a central information processing device and the like, a program memory 802, a data memory 803, a display unit 804, a communication interface 805 of Bluetooth (registered trademark) 71, and a communication interface 806 of Wi-Fi (registered trademark) 72. The information processing unit 801 functions based on an OS 8021 and an application program 8022 that are stored in the program memory 802, and can display, in the display unit 804, data 8031 or 8032 stored in the data memory 803, and exchange data using the communication interfaces 805 and 806. The information processing device 8 is constituted by a tablet terminal, and includes standard functions that are mounted on a tablet terminal such as a camera, a flash, a touch panel sensor, and an antenna, in addition to the above-described constituent elements.

In the individual health information system configured as described above, it should be noted that, in the present embodiment, while some area of the data memory 503 of the mobile communication terminal 5 on the owner 1 side is used as storage means for recording health information 61 of the member, the information processing unit 501 and receiving means 508 are normally intermittently activated by a dedicated application program 5023 stored in the program memory 502. Also, it should be noted that the information processing unit 501, upon the receiving means 508 receiving a voice 70 that is a predetermined activation sign from the outside, transmits the health information 61 to the information processing device 8 by short range wireless communication using the communication interface 505 of Bluetooth (registered trademark) 71. In the information processing device 8, when the communication interface 805 has received the health information 61, the information processing unit 801 displays the health information 61 on the display unit 804, and stores the health information 61 in the data memory 803 as mirror data 8033, by a dedicated application program 8023 stored (pre-installed) in the program memory 802. Therefore, the communication interface 805 of the information processing device 8 is also normally intermittently activated by the dedicated application program 8023.

With this, even in a situation in which a wide area wireless communication network formed by the mobile phone channel 73, that is, the so-called Internet cannot be used, health information 61 that is the same as that stored in the mobile communication terminal 5 on the owner 1 side can be mirrored to the information processing device 8 on the medical professional 9 side, as shown in FIG. 1. In FIG. 1, the selection screen of the health information 61 is illustrated on the display unit 504 of the mobile communication terminal 5 as well, but if the owner 1 is in a serious condition, it is difficult for the owner 1 to confirm the information, and therefore whether or not the health information 61 is displayed on the display unit 504 in a state of the data being mirrored can be predetermined by a setting or the like.

Specifically, in the mobile communication terminal 5, when turning ON of the function is selected by a later-described setting operation, as in the case of an application program (5022) of a pedometer, the dedicated application program 5023 is activated all the time in the individual health information system, and the system waits for a predetermined activation sign such as calling from the medical professional 9. Also, when the receiving means 508 receives the activation sign issued by the medical professional 9 in a predetermined form, the information processing unit 501 transmits the health information 61 recorded in the data memory 503 to the communication interface 805 of the information processing device 8 from the communication interface 505. When the communication interface 805 receives the health information, the information processing device 8 displays the health information by display means 804.

The short range wireless communication is not limited to Bluetooth (registered trademark) 71 used by the abovementioned communication interfaces 505 and 805, and another short-range wireless LAN such as Wi-Fi (registered trademark) 72 used by the communication interfaces 506 and 806 may be used. However, Bluetooth (registered trademark) 71 has low power consumption in standby due to a LE (Low Energy) technology, and is preferable in a case where the standby time is very long and it is not known when communication will be performed, as in the present embodiment.

By adopting this configuration, the health information 61 of predetermined members that include the owner 1 and family members, depending on the case, that is recorded in the mobile communication terminals such as a smartphone can be mirrored to the information processing device 8 such as a smartphone or a tablet terminal that is possessed by a medical professional 9 such as a paramedic or a doctor that performs treatment on a member when the member is in poor physical condition. That is, in many cases, the health information 61 of the member recorded in the mobile communication terminal 5 such as a smartphone of the owner 1 may be locked by biometrics authentication by the smartphone itself or a password, but when a voice 70 that is a predetermined activation sign is received from the outside, the lock is canceled by the dedicated application program 5023, and the health information 61 is forcibly mirrored to the information processing device 8 such as a smartphone or a tablet terminal that is possessed by the medical professional 9. Note that whether or not mirroring is possible when a plurality of the information processing devices 8 are present depends on the method of short range wireless communication to be used, that is, whether one-to-one connection is made in time series, or one-to-many connection is made at the same time.

With this, even if the name of the owner 1 is unknown, or if the health information 61 of the member cannot be obtained from his or her family doctor's hospital, on the medical professional 9 side such as an ambulance 91 or an ambulance helicopter due to the large-scale wireless communication network such as the mobile phone channel 73 being unstable, the health information 61 can be obtained directly, reliably, and stably by short range wireless communication (locally) with the mobile communication terminal 5 such as the smartphone of the owner 1. With this, the medical professional 9 can perform treatment quickly and appropriately. Specifically, not limited to the ambulance 91, in a large-scale disaster or accident in which the communication infrastructure is likely to fail, the health information 61 can be obtained on site until the batteries of the mobile communication terminal 5 and the information processing device 8 run out.

Here, the receiving means 508 can be realized by a microphone, speech recognition software, and the like, and a so-called AI speaker technology that is widely installed in the smartphone for information retrieval can be used. The activation sign is calling by the voice 70 of a predetermined word that is spoken by a medical professional 9. For example, “mirror health information”. When the voice 70 is recognized by the receiving means 508, the information processing unit 501 reads out the health information 61 recorded in the data memory 503 and transmits the health information 61 from the communication interface 505, in accordance with the dedicated application program 5023. When the microphone and the speech recognition software are used as the receiving means 508, a tone from a transmitter or the like may be used, and is not limited to the voice. For example, “peep” or “blip” of the push tone of a telephone may be used. In the case of a noisy ambulance helicopter, a sound that is easily discernable is preferable.

Also, regarding the activation, a configuration may be adopted in which, since the dedicated application program 8023 is stored (pre-installed) in the program memory 802 of the information processing device 8, the application program 8023 activates activation signal generating means 808, and a generated activation signal is applied to the information processing unit 501 as the activation sign via the communication interfaces 805 and 505 of Bluetooth (registered trademark) 71. With this, when the medical professional 9 locally issues an activation signal, from the information processing device 8 such as a smartphone or a tablet terminal, by Bluetooth (registered trademark) 71 toward the mobile communication terminal 5 such as the smartphone of the owner 1, the medical professional 9 can locally acquire the health information 61 by the same Bluetooth (registered trademark) 71.

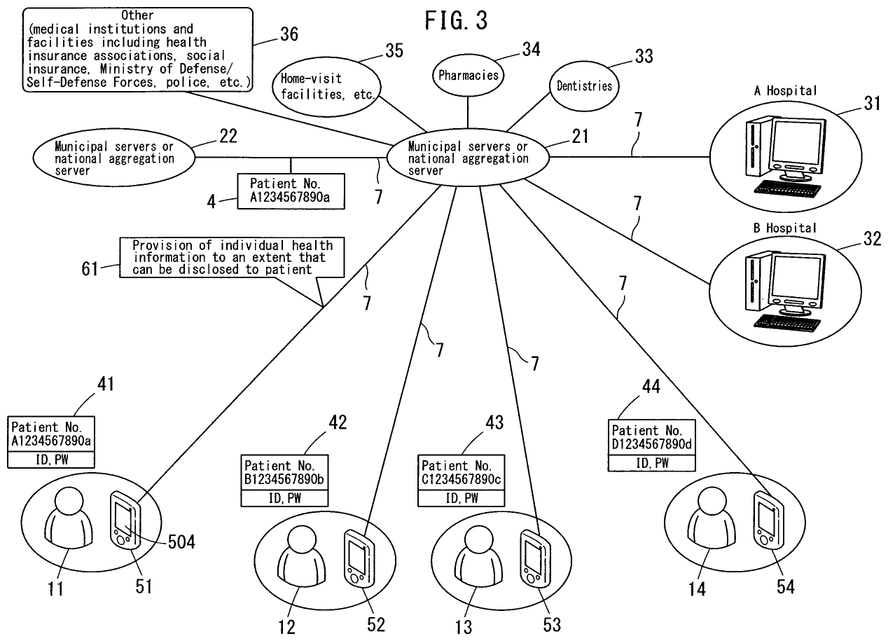

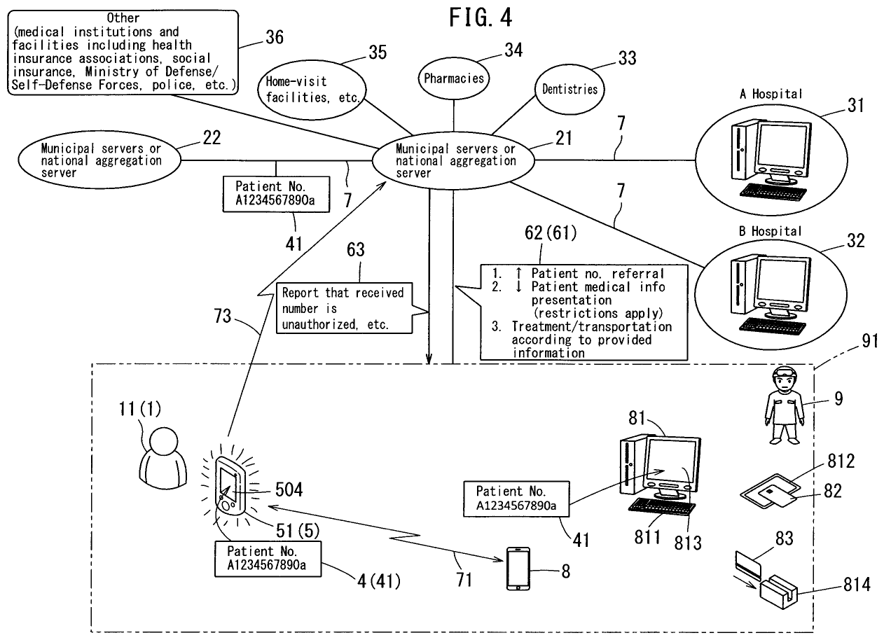

FIGS. 3 and 4 are block diagrams illustrating the overall configuration of the individual health information system according to one embodiment of the present invention. FIG. 3 shows a state of the owner (examinee, patient) 1 under normal conditions. FIG. 4 shows a state in which the owner 1 is transported in an emergency situation. Note that in the above description, the owner 1 is described as the owner of the mobile communication terminal 5 constituted by a smartphone, but in the following, the owner 1 may be an examinee who receives an examination, or a patient that is transported in an emergency situation, and although a term that is appropriate in each situation will be used, the same reference sign will be used.

Referring to FIGS. 3 and 4, the individual health information system of the present embodiment aims to enhance convenience, as a result of health information 61 of individual examinees (patients) 11 to 14 (hereinafter, denoted by reference sign 1 when referred to collectively) being aggregated and managed in aggregation servers of management centers 21 and 22 (hereinafter, denoted by reference sign 2 when referred to collectively). The management center 2 need only be installed as appropriate, and may be installed in each administrative district or centrally manage the entire country. For example, the management centers 2 are installed in enterprises that have commercialized in advance, or competent institutions and contract institutions of relevant government offices such as third sectors, incorporated associations, and foundations.

Specifically, in this individual health information system, for each of a plurality of examinees 1, health information 61 that includes, as appropriate, a current medical record, medication information and the like, and also a past clinical history, chronic conditions and the like for each examinee 1 collected in a plurality of medical institutions 31 to 36 (hereinafter, denoted by reference sign 3 when referred to collectively) are aggregated and held in association with identification information 41 to 44 (hereinafter, denoted by reference sign 4 when referred to collectively), which are unique patient numbers assigned in advance to each examinee 1, in the management center 2. The health information 61 can be viewed based on a predetermined rule in response to the request from the medical professional 9, the examinee 1 him/herself, or a family member. In this individual health information system, medical test images are, for example, exchanged in the form of data conforming to DICOM (Digital Imaging and Communication in Medicine), which is a standard relating to medical test images and communication thereof, and are also held in the management center 2. In the management center 2, a database that actually holds the health information can be realized by an aggregation server such as NAS (Network Attached Storage).

In the example of FIGS. 3 and 4, the medical institutions 31 and 32 are general hospitals, the medical institution 33 is a dentistry, the medical institution 34 is a pharmacy, the medical institution 35 is a home-visit nursing care facility, and the medical institution 36 is another healthcare-related facility (health insurance society, social insurance, Ministry of Defense (Self-Defense Force), police force, etc.). FIG. 3 shows an example in which the identification information 41, 42, 43 and 44, namely, “A1234567890a”, “B1234567890b”, “C1234567890c” and “D1234567890d”, are respectively allocated to the examinees 11, 12, 13, and 14.

Also, the examinees 11 to 14 access, at an appropriate timing such as after reception by the medical institutions 3, the aggregation server of the management center 2 from a wireless transceiver 507 via the mobile phone channel 73, using the application program 5023 of mobile communication terminals 51 to 54 (hereinafter, denoted by reference sign 5 when referred to collectively) such as a smartphone or tablet terminal in their possession, and, through input of the identification information 4 and a predetermined login ID and password, it becomes possible for health information 61 of oneself, a family member, or the like to be acquired, recorded in storage means of the data memory 503, viewed in the display unit 504 or presented at the medical institution 3. The medical institution 3, the management center 2, and the mobile information terminal 5 are connected to each other via a network 7 such as the mobile phone channel 73 or an Internet connection.

The individual health information system of the present embodiment is thus extremely effective for the examinee 1 or a family member going to a new medical institution (31, 32, 36) as an outpatient taking the health information 61 of the examinee 1 with him or her in order to get a second opinion or the like, and for a local insurance society or medical association which is not shown checking that there is no duplication of medication and no errors in the medical billing claim. Also, troublesome and time-consuming tasks such as getting a referral, in order for the other hospital (31, 32, 36) or clinical department to obtain previous health information, can be dispensed with.

Note that the predetermined rule for viewing of the health information 61 is for actions such as restricting the health information 61 to a level viewable by a doctor, to a level viewable by a pharmacy, and to a level viewable by the examinee 1 himself or herself or a family member. Specifically, the rule may be such that all information can be viewed by a clinical department in a hospital (31, 32, 36), but a pharmacy (34) or the examinee 1 is unable to view information relating to the progressive stage, and information on communicable diseases (HIV, hepatitis, etc.) that is required at the time of treatment can only be viewed by a hospital (31, 32, 36). The identification information 4 is an ID number or the like that is allocated one to each examinee, and is not allocated for every medical institution (31, 32, 36) unlike that on a patient registration card, and also differs from the above login ID and password that the examinee 1 is able to arbitrarily set in advance.

Also, it should be noted that, in the individual health information system of the present embodiment, the dedicated application programs 5021 and 8021 are stored (pre-installed) in the program memories 502 and 802 of the mobile communication terminal 5 and the information processing device 8, and therefore bidirectional communication through Bluetooth (registered trademark) 71 is possible, and selection of the health information 61 is possible from the information processing device 8 side. Of course, all of the health information 61 stored in the storage means of the data memory 503 may be collectively transmitted in response to an activation sign such as the voice 70, but in this case, it is possible that it will take time beforer a medical professional 9 can confirm desired health information in the information processing device 8. In view of this, this selection is enabled.

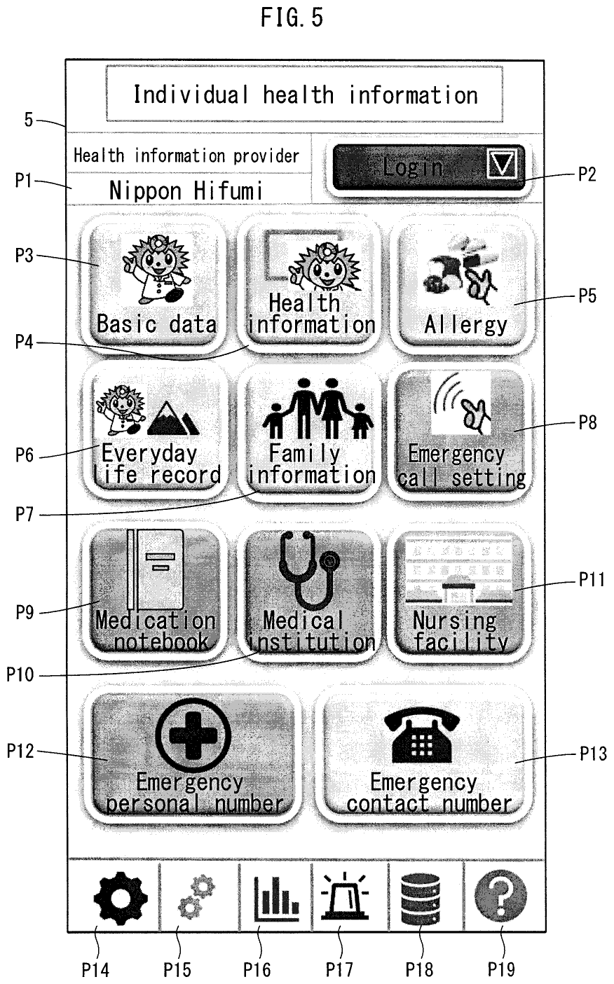

FIG. 5 is an example of a top screen for henceforth selecting desired health information. When the dedicated application program 5023 is installed in the mobile communication terminal 5 constituted by a smartphone or the like, and an icon of a standby display of the display unit 504 is clicked, this top screen is displayed, and also this top screen is displayed in the display unit 804 of the information processing device 8 as a first operation of the abovementioned mirroring operation, which is as shown in FIG. 1.

In the top screen, a name P1 and a login button P2 are displayed, and in addition thereto, various icons P3 to P19 are displayed. Unless the ID and password are input by operating the login button P2 and authenticated, the operations of the remaining icons P3 to P13 are invalidated. The color of the login button P2 changes to indicate the state of the receiver 1, such as the period from the most recent hospital visit or house visit, for example, by being red if the period is one week or less, yellow if the period is three weeks or less, green if the period is one month or less, and colorless if the period is over one month.

The icon P3 is for opening a basic data screen, and by operating the icon, kana/Chinese characters of the name, birth date, blood type, address, telephone number, mail address, ID of this individual health information system, ID of the medication notebooks, face photo, address and telephone number of the emergency contact, address and telephone number of the workplace, and the like can be confirmed.

The icon P4 is for opening a screen of health information and treatment history, and by operating the icon, desired information can be extracted and displayed, through successive layers, from the treatment and examination histories, diagnostics results, test results of blood and the like, medical records, test images, and the like. The icon P5 is for opening a screen regarding allergies, and allergies and the diagnostics regarding medicine, environment, food, pollen, and the like, and treatment records can be confirmed. The icon P6 is for opening a screen regarding everyday life situation, and the preferences of cigarette, alcohol, and the like, and the periods of intake can be confirmed. In this everyday life situation, a configuration may be adopted in which data such as a blood pressure and pulse detected by a separate biosensor is imported, compiled as appropriate, and the result is displayed.

The icon P7 is for opening a screen regarding the family information, and personal information regarding each family member can be confirmed, similarly to the operation of the icon P3, in a similar manner to turning over pages. The icon P8 is a transmission button to an emergency call destination, and by operating this icon, a telephone call can be made to a pre-registered call destination such as a family member, friend, or doctor in charge. The icon P9 is for opening a screen regarding medication histories such as a medication notebook, past prescriptions corresponding thereto, and the medical institution that has issued prescriptions, dispensing pharmacy and the location thereof, prescriptions, and the like can be confirmed. The icon P10 is for showing information regarding the medical institution that the receiver 1 visits and nearby medical institutions, and the hospital name, location, doctor name, and the like can be confirmed. By operating the icon P9 regarding the medication notebook, and the icon P10 regarding the medical institutions, the information regarding the examinee 1 as described above can be of course conformed, and in addition thereto, nearby pharmacies and medical institutions can also be introduced from the current location information of the information communication terminal 5 such as a smartphone.

The icon P11 is for displaying information regarding a nursing facility that is visited for rehabilitation or in which the examinee 1 is actually accommodated, if relevant. The icon P12 is for confirming identification information 41, 42, 43, or 44 of the individual health information such as the abovementioned “A1234567890a”, and by operating this icon, the identification information is displayed in the display unit 504 in a large size, and the medical professional 9 can confirm the information, as will be described later. The icon P13 is for displaying telephone numbers of emergency contacts such as an ambulance (firefighting), the police when an accident occurs, and a wrecker arrangement, and also for making a call by a button operation.

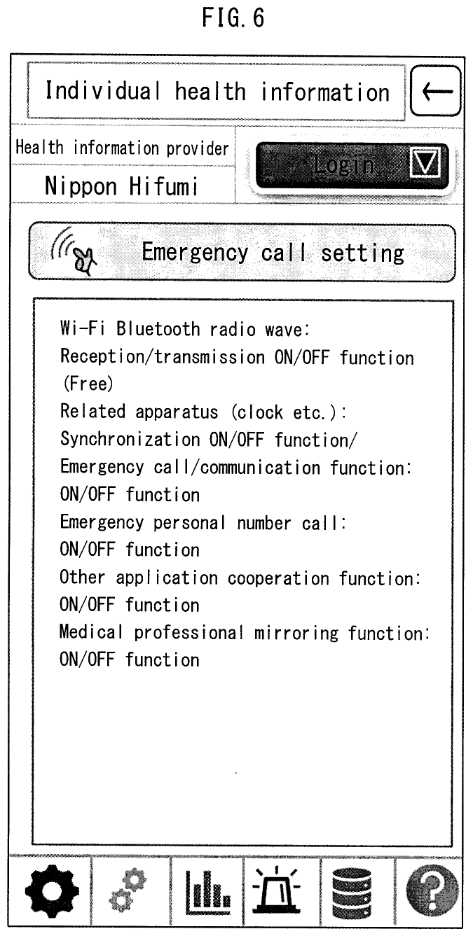

The icons P14 to P19 in the lower portion are buttons for settings and operation confirmation, and by operating the icon P14 for setting, the setting screen in FIG. 6 is displayed. In the setting, ON/OFF of the communication interface 505 of Bluetooth (registered trademark) 71 and the communication interface 506 of Wi-Fi (registered trademark) 72, ON/OFF of an emergency call by the icon P8, ON/OFF of an automatic transmission function of each of the identification information 41, 42, 43, and 44, which will be described later, and ON/OFF of the mirroring function of the abovementioned health information 61, and the like can be set.

In the individual health information system configured as described above, by issuing a voice 70 or an emitting sound that is an activation sign, or transmitting an activation signal of Bluetooth (registered trademark) 71 that is an activation sign from an information processing device 8, a medical professional 9 can mirror the health information 61 of the examinee 1 to an extent recorded in the data memory 503 of the information communication terminal 5 to the information processing device 8 by short range wireless communication of Bluetooth (registered trademark) 71 that is available only inside an ambulance 91, confirm the health information 61, and utilize it to perform first-aid measures on the examinee 1, as described above.

However, the health information 61 recorded in the data memory 503 may not include all of the health information of the examinee 1. Specifically, in a case of a young and healthy person having a infrequent visiting history to medical institutions, the health information of the information communication terminal 5 need only be confirmed. On the other hand, in a case of an examinee having a long visiting history such as an elderly person who often contracts diseases, all of the health information 61 may not be stored in the information communication terminal 5. In view of this, in this individual health information system, a medical professional 9 can utilize the health information 61 on the aggregation center 2 by acquiring the pieces of identification information 41, 42, 43, and 44 of the individual health information such as “A1234567890a” by operating the icon P12.

Specifically, utilization thereof involves a medical professional 9 acquiring the identification information 4 of an examinee (patient) 1 in a place where the examinee 1 is accommodated such as the inside of an ambulance 91 or an ambulance helicopter, with reference to FIG. 4. Acquisition of the identification information 4, in the case where the examinee (patient) 1 is conscious and remembers the identification information 4, is carried out by the medical professional 9 asking the examinee (patient) 1 and inputting (typing in) the identification information 4 provided by the examinee (patient) 1 with a keyboard 811 of a personal computer 81 in a manner that will be described later. Alternatively, if the examinee (patient) 1 does not remember or the examinee (patient) 1 is unconscious, the identification information 4 can be acquired by operating the icon P12 in the mirrored state described above. In the inside of the ambulance 91 or the ambulance helicopter, the terminal is not limited to the personal computer 81 that is fixedly installed, and a movable tablet terminal such as the information processing device 8 may be used, or both of them may be used.

In this individual health information system, when the identification information 4 is thus obtained, the medical professional 9 accesses the aggregation server of the management center 2, and accesses (downloads) the health information 61 of the examinee (patient) 1, as denoted by reference sign 62, with the personal computer 81, using his or her own ID, password, or the like. The ID and password are used in authenticating whether the medical professional 9 has the credentials to access (download) the health information 61 of the examinee (patient) 1. Input of the ID and password may be performed with the keyboard 811, but may also be carried out by an IC card 82 or a magnetic card 83 that the medical professional 9 possesses being brought close to, in contact with, or inserted into a card reader 812 or 814, or the like, and information being read.

In cases such as where the identification information 4 of the examinee (patient) 1 is not correct (the corresponding examinee does not exist, i.e., the identification information 4 does not exist), or where the ID or password of the medical professional 9 is not correct, that is, the password is incorrect or has been changed, or when the ID is no longer valid, the aggregation server of the management center 2 sends a reply indicating that this information is incorrect to the personal computer 81, as denoted by reference sign 63. The ID of the medical professional 9 is not applied for individually, for example, but rather the affiliated organization such as the firefighters association or the contract institution of this system collectively applies to a system administrator and receives issuance.

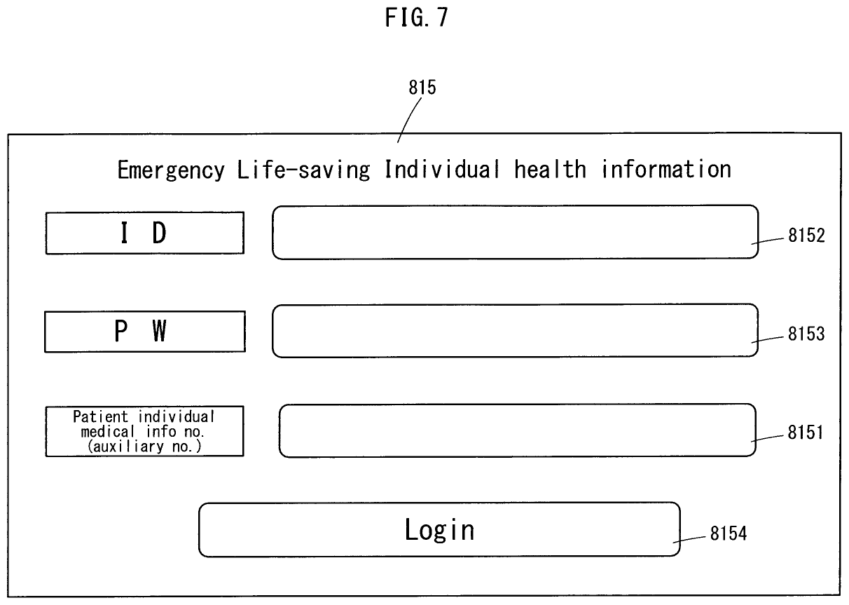

FIG. 7 is a diagram illustrating one example of an input screen 815 of a personal computer 81 for the medical professional 9 accessing (downloading) the health information 61 of the examinee (patient) 1, and the medical professional 9 can access the aggregation server by inputting the identification information 4 as the patient individual health information number in an input field 8151, inputting his or her own ID into an input field 8152, inputting the password in an input field 8153, and clicking a login button 8154. When the IC card 82 or magnetic card 83 is used, the input fields 8152 and 8153 are automatically filled.

When authentication of the medical professional 9 is performed by the aggregation server and the health information 61 of the examinee (patient) 1 exists, the health information 61 is downloaded and displayed on a display screen 813 of the personal computer 81 which is display means.

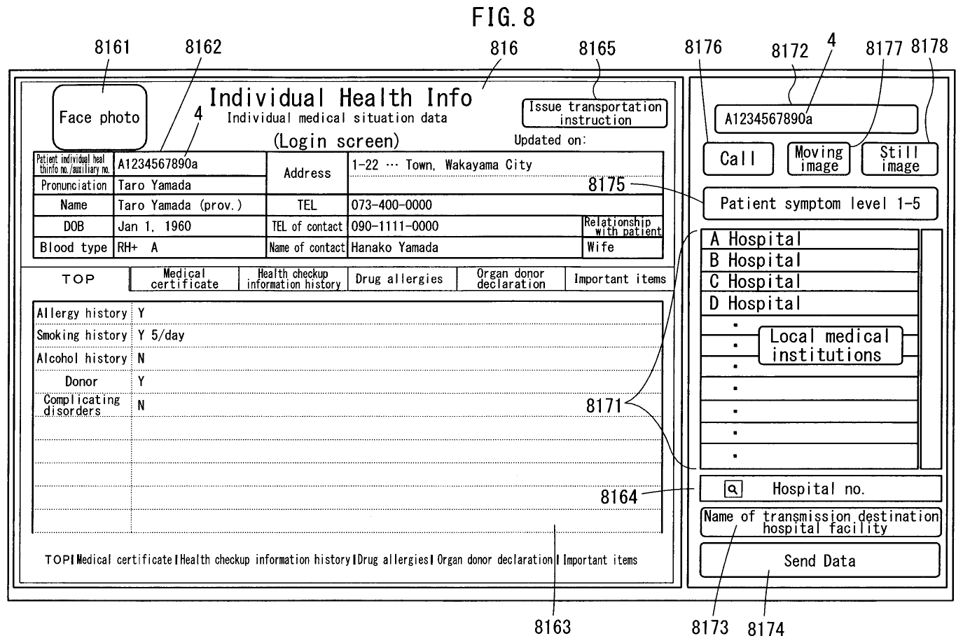

FIG. 8 is a diagram showing an example of a display screen 816. Broadly, a face photo 8161 and personal information 8162 are displayed in upper left fields of the display screen 816. Accordingly, personal authentication of the examinee (patient) 1 is performed with the identification information 4 as aforementioned, and the medical professional 9 is able to again perform personal identification of the examinee (patient) 1 with this face photo 8161. The personal information 8162 includes the identification information 4, name, birth date, blood type, address, telephone number, contact, and the like. An instruction button 8165 is a button for requesting selection of a receiving medical institution by the local fire brigade headquarters (Tel: 119).

A lower left field 8163 of the display screen 816 is in a multiple page configuration, and basic information (TOP) such as allergies and preferences, medical certificates of previous consultations, health checkup results, an organ donation declaration and the like are displayed. A right-side field 8164 of the display screen 816 is used for coordinating with the medical institution serving as the transportation destination, and includes items used in exchanges for preparing to receive the examinee (patient) 1, which will be described below, in addition to medical institutions serving as receiving destination candidates being displayed in candidate order and contact details and the like being displayed.

Specifically, in the right-side field 8164, medical institutions serving as receiving destination candidates are displayed in candidate order in a list 8171. Also, the identification information 4 is displayed in an upper field 8172. The identification information 4 in the upper field 8172 and the health information 61 in left-side fields can be transmitted to the hospital designated in a field 8173 by clicking a send button 8174. This can be useful in helping the hospital prepare to receive the examinee (patient) 1.

In this individual health information system, a plurality of icons 8175 to 8178 for conveying the condition of the examinee (patient) 1 to the medical institution are also provided in the right-side field 8164 that is used for coordinating with the medical institution serving as the transportation destination. In icon 8175 portion, the result of a medical professional 9 determining, over five levels, for example, the state of the examinee (patient) 1 determined through predetermined measures, is displayed, and by clicking on this icon 8175, the determination result is transmitted to the medical institution (henceforth, receiving medical institution) that is scheduled to ultimately receive the examinee (patient) 1 designated in the field 8173. In the case where the state of the examinee (patient) 1 is not easily conveyed with images or moving images which will be described later, this configuration enables the state of the examinee (patient) 1 to be quickly conveyed to the receiving medical institution.

Also, by clicking on the icon 8176, a handsfree call is possible, enabling a medical professional 9 to seek instructions from a doctor in the receiving medical institution. Furthermore, by clicking on the icon 8177, a moving image shot with the camera inside the ambulance 91 is transmitted to a display device of the receiving medical institution such as 31, for example. By clicking on the icon 8178, a still image of higher definition than the moving image, for example, in which the affected area or injury is shown in close-up is transmitted to the display device of the medical institution 31, for example. The personal computer 81 serves as transmission means of these images and audio. Also, the management center 2 that transfers these images and audio to the display device of the receiving medical institution 31 serves as relay means, but such relay is desirably performed by other relay means, without passing through the aggregation server of health information 61.

In this individual health information system, first, the health information 61 locally recorded in the mobile communication terminal 5 can be mirrored to the information processing device 8 so as to be able to be remotely (selectably) operated in response to calling by the voice 70, a tone from a transmitter, or an activation signal from the activation signal generating means 808 through Bluetooth (registered trademark) 71, as described above. Moreover, when the identification information 4 can be obtained and the network 7 such as the mobile phone channel 73 and the Internet connection can be used, detailed health information such as higher definition images can be obtained from the aggregation center 2, a large amount of information can be collectively displayed using a large screen of the display screen 816 of the personal computer 81, as shown in FIG. 8, and can communicate with medical institutions 3. In this way, the health information 61 of each individual can be effectively used in emergency situations such as a wound, disease, accident, or disaster, and a medical professional 9 can easily take an appropriate measure, and the lifesaving rate can be improved. Specifically, as a result of the health information 61 is mirrored from the mobile communication terminal 5 to the information processing device 8, as described above, even in a case where the network 7 cannot be used due to a large-scale disaster, the health information 61 can be utilized for an appropriate treatment.

Here, in order to cause the display unit 504 to display the identification information 4 by operating the icon P12, a medical professional 9 needs to locate the mobile communication terminal 5. In this case, when the examinee (patient) 1 cannot handle the situation such as a case where the examinee (patient) 1 is unconscious, there are cases where it is not known whether or not the examinee (patient) 1 has a mobile communication terminal 5, and even if he or she does, the location thereof is unknown. In view of this, the information processing means 501 displays the identification information 4 in the display unit 504, and further includes notifying means 509 for performing vibration in order to notify the medical professional 9 of the location of the mobile communication terminal 5, when an AI speaker has responded to calling by the voice 70 or a tone from a transmitter that is an activation sign, or the communication interface 71 has received an activation signal. The method of notification may be appropriately selected from methods such as emitting sound, vibration, and emitting light.

With this, the location of the mobile communication terminal 5 can be easily determined. Note that when the mobile communication terminal 5 is operated from the information processing device 8 by mirroring and remote operations, although the mobile communication terminal 5 need not be located, it is desirable that the notifying means 509 makes a response, because it is not known whether or not the examinee (patient) 1 possesses the mobile communication terminal 5, and even in the case of possessing, whether or not the mobile communication terminal 5 is compatible with this individual health information system.

Also, in the abovementioned description, the ambulance 91 is illustrated as the place where the examinee (patient) 1 is accommodated in FIGS. 1 and 4. Of course, there are cases where the examinee (patient) 1 is accommodated in a hospital that is the transportation destination, and the wide area communication network 7 (73) can be used in many of these cases. However, in rare cases, the medical professionals 9 act in situations of large-scale disasters and accidents, and in many of these cases, the medical professionals 9 need to act in difficult conditions. For example, there are cases where the place in which treatments and procedures are performed is a public facility such as a school or a public hall, or inside a temporary tent. Also, the wide area communication network 7 (73) may not be available due to the base station 25 (refer to FIG. 2) of mobile phones being broken or power supply being insufficient. In such cases, as described above, it is extremely useful that the health information 61 recorded in a mobile communication terminal 5 is mirrored to an information processing device 8, and is utilized for performing treatments and procedures.

In addition thereto, it is preferable that the contents of treatments and procedures that are briefly summarized by only characters and input to an information processing device 8 by a medical professional 9 are transmitted to a mobile communication terminal 5 via the communication interface 71, or the contents are directly input to the mobile communication terminal 5, and the contents are added to the health information 61 of the examinee (patient) 1. With this, a simple medical record and a tag move (travel) along with the examinee (patient) 1, and therefore if a doctor has input the contents and a nurse takes over, for example, the nurse can quickly grasp the conditions and appropriately perform treatments and the like. In the cases of the large-scale disaster and accident, there will be a large number of examinees (patients) 1, and therefore such grasping of conditions is extremely effective.

Thereafter, when the wide area communication network 7 (73) is restored, the mobile communication terminal 5 uploads the additional information to a server of a management center 2 along with the identification information 4, and the additional information is shared on the server side as well, as shown in FIG. 4.

Note that it is quite a task for an examinee (patient) 1 or the like to input health information 61 such as tests, treatment, procedures, and medication to a mobile communication terminal 5 such as a smartphone, and perform uploading, as described above. Specifically, it is not realistic for a diagnostic image or the like to be shot by a built-in camera and imported to the smartphone. Therefore, it is desirable that the amount of data to be uploaded to the server of the management center 2 by a mobile communication terminal 5 is to the extent of the additional information, which is small data. Realistically, every time the health information 61 utilized as an electronic health record is newly input to a server of a medical institution 3 and is updated, on the medical institution 3 side, the health information 61 is copied from the server of the medical institution 3 to a server of the management center 2, and furthermore the health information 61 is transmitted to a mobile communication terminal 5 of the examinee (patient) 1 through the wide area wireless communication network 7 such as the mobile phone channel 73, and as a result, the health information 61 in the data memory 503 is similarly updated. Accordingly, the examinee (patient) 1 can record the latest health information 61 in the mobile communication terminal 5 without performing troublesome input work.

The individual health information system of the present invention is an individual health information system that is configured to be provided with a mobile communication terminal and an information processing device, and is characterized as follows. The mobile communication terminal is for recording health information of members related to its owner and including at least the owner, and the mobile information terminal is configured to be provided with storage means for recording the health information, receiving means for receiving a predetermined activation sign from the outside, a first communication interface for performing short range wireless communication with the information processing device, and information control means for, upon the receiving means receiving the predetermined activation sign, transmitting the health information stored in the storage means from the first communication interface to the information processing device. Moreover, the information processing device is possessed by a medical professional that performs treatment when a member is in poor physical condition, and is configured to be provided with a second communication interface, and display means for, upon the second communication interface receiving the health information, displaying the health information.

The abovementioned configuration is for health information of predetermined members that include an owner of a smartphone and the family members, depending on the case, the health information being recorded in a mobile communication terminal such as the smartphone of the owner, to be mirrored to an information processing device such as a smartphone or a tablet terminal that is possessed by a medical professional such as a paramedic or a doctor that performs treatment when the members are in poor physical condition. Specifically, in the mobile communication terminal, an application program dedicated to this individual health information system is activated all the time, as in the case of an application program of the pedometer, and the system waits for a predetermined activation sign such as calling from a medical professional or a specific tone. Also, in the mobile communication terminal, when the receiving means has received the activation sign generated by the medical professional, the information control means transmits health information stored in the storage means to the information processing device from the first communication interface by short range wireless communication. In the information processing device, when the second communication interface has received the health information, the display means displays the health information. The short range wireless communication can be realized by a short range wireless LAN such as so-called Wi-Fi (registered trademark, IEEE 802.11 standard) or, Bluetooth (registered trademark, IEEE 802.15 standard).

Therefore, although the health information of members recorded in a mobile communication terminal such as a smartphone of an owner may be, of course, locked by biometrics authentication by the smartphone itself or a password, when a predetermined activation sign is received from the outside, the lock is released/canceled, and the health information is forcibly mirrored to an information processing device such as a smartphone or a tablet terminal that is possessed by a medical professional. With this, even if the name of the owner is unknown, or even in a case where the health information of the members cannot be obtained from his or her family doctor's hospital or the like on the medical professional side such as an ambulance or an ambulance helicopter due to the large-scale wireless communication network such as a mobile phone channel being unstable, the health information can be (locally) acquired directly, reliably, and stably by short range wireless communication with a mobile communication terminal such as a smartphone of the owner, and as a result, treatment can be performed quickly and appropriately.

Also, in the individual health information system of the present invention, the activation sign is calling by a predetermined voice made by the medical professional or an emission of a predetermined tone of a transmitter.

According to the above configuration, as a result of a medical professional uttering a voice such as a predetermined word serving as an activation sign, or generating a predetermined tone by a transmitter toward a mobile communication terminal such as a smartphone of the owner, the health information can be (locally) acquired from the mobile communication terminal such as the smartphone of the owner by short range wireless communication.

Furthermore, in the individual health information system of the present invention, the activation sign is realized by transmitting an activation signal generated by activation signal generating means provided in the information processing device from the second communication interface by the short range wireless communication, and the first communication interface of the mobile communication terminal receiving the activation signal.

According to the above configuration, as a result of a medical professional (locally) generating the activation signal from an information processing device such as a smartphone or a tablet terminal toward a mobile communication terminal such as a smartphone of the owner by short range wireless communication, the information processing device further (locally) acquires the health information by the short range wireless communication.

Also, in the individual health information system of the present invention, the first and second communication interfaces can perform bidirectional communication, and the information processing device includes selecting means of the health information, when receiving of the health information from the mobile communication terminal becomes possible, the information processing device transmits the result of selection by the selecting means to the information control means from the second communication interface via the first communication interface, and the information control means reads out the health information that is the selection results from the storage means and transmits the health information to the information processing device.

According to the above configuration, when mirroring of health information recorded in a mobile communication terminal to an information processing device becomes possible (activated), furthermore, the information processing device remotely control the mobile communication terminal so as to be able to retrieve necessary information.

Therefore, for example, a member who needs to read out health information can effectively retrieve only the necessary information needed for a medical professional to perform treatment when a member is in poor physical condition, such as the patient being which of the family members, a clinical history for performing diagnostics, or a medication history for performing treatment.

Furthermore, in the individual health information system of the present invention, when transmission of health information to the information processing device becomes possible, new health information can be input to the mobile communication terminal using its input means, and the information control means records the input health information to the storage means, and reads out the health information and causes display means to display the health information in response to an operation made on the input means.

According to the above configuration, when mirroring of health information recorded in a mobile communication terminal to an information processing device becomes possible (activated), new health information can be input to the mobile communication terminal using its input means, and the information control means records the input health information to the storage means.

Therefore, even in a situation in which health information cannot be written into a mobile communication terminal from an information processing device using a wide area network such as a mobile phone, the medical professional can directly write health information into the mobile communication terminal by operating a touch panel or the like. With this, in a situation in which many persons are injured due to a large-scale disaster, accident, or the like, as a result of a medical professional recording a diagnostics result or symptoms in a mobile communication terminal even in a brief summary, a medical professional who next sees (takes over) the health information can immediately grasp the situation.

Also, the individual health information system of the present invention is configured to be provided with a server that aggregates and holds health information of each of a plurality of examinees, who are the members, that are collected in a plurality of medical institutions in association with unique identification information assigned in advance to the examinee, and enables viewing of the health information of the examinee in the mobile communication terminals and information processing device based on a predetermined rule, in response to a request of a medical professional and/or the examinee, a plurality of the mobile communication terminals and information processing devices, and a wide area wireless communication network that connects the server, the mobile communication terminals, and the information processing devices. The receiving means, upon receiving a predetermined activation sign from the outside, displays unique identification information assigned in advance to the examinee, and the information processing device acquires the aggregated health information by accessing the server with the unique identification information assigned in advance to the examinee through the wide area wireless communication network.

According to the above configuration, an individual health information aggregation system can be realized by aggregating and holding health information including current medical records and medication information for each of a plurality of examinees collected in a plurality of medical institutions and also a past clinical history or the like as appropriate in a management center or the like, and enabling viewing of the health information of the examinee, in response to a request from the medical professional, the examinee or a family member or the like based on a predetermined rule. This individual health information aggregation system is extremely effective for an examinee or a family member going to a new medical institution as an outpatient taking the health information of the examinee with him or her in order to get a second opinion or the like, for a local insurance society or medical association checking that there is no duplication of medication and no errors in the medical billing claim. Also, troublesome and time-consuming tasks such as a getting referral, in order for the other hospital or clinical department to obtain the previous health information, can be dispensed with.

The predetermined rule for viewing the health information is for actions such as restricting the health information to a level viewable by a doctor, to a level viewable by a pharmacy, and to a level viewable by the examinee 1 himself or herself. The identification information is an ID number or the like that is allocated one to each examinee, and is not allocated for every medical institution unlike that on a patient registration card.

The present invention is configured such that medical professionals such as paramedics, doctors and nurses who perform first-aid measures are able to carry out appropriate measures and advance preparations utilizing health information that is associated with the unique identification information assigned in advance to the examinee, in the accommodation place of the examinee such as the inside of an ambulance or at a hospital serving as the transportation destination when the examinee is in poor physical condition, such as when transported in an emergency situation, for example, utilizing such as the individual health information aggregation system. Specifically, first, the identification information of the examinee needs to be acquired, and the receiving means, upon receiving a predetermined activation sign from the outside, e.g., calling of the first communication interface from the second communication interface, displays the unique identification information assigned in advance to the examinee. The information processing device that has acquired the identification information as a result of a medical professional reading out the identification information and inputting it thereto, or by utilizing the receiving means and the short range wireless communication, accesses the server though a wide area wireless communication network such as a mobile phone channel, and acquires the health information aggregated in association with the identification information.

That is, the information processing device does not acquire the health information itself of the examinee that is recorded in a mobile communication terminal such as a smartphone of the owner through the abovementioned short range wireless communication, and instead separately acquires detailed health information from a server installed at the aggregation center or the like using identification information such as an ID number acquired from the mobile communication terminal. Also, information that can be viewed only by a medical professional and is restricted from being viewed by the examinee is also obtained, and more appropriate treatment and procedures can be performed.

Moreover, in the individual health information system of the present invention, the place where the member is accommodated when the member is in poor physical condition is the inside of an ambulance or an ambulance helicopter, and a medical professional that performs the treatment is a paramedic and/or a doctor.

According to the above configuration, even when a wide area wireless communication network cannot be used or is unstable, the health information can be mirrored from a mobile communication terminal such as a smartphone of the owner by short range wireless communication, and can be directly shared by paramedics and doctors, and therefore the configuration can be adapted to a moving ambulance or ambulance helicopter as the accommodation place of the examinee who is in poor physical condition.

Also, in the individual health information system of the present invention, the mobile communication terminal, when the receiving means receives an activation sign, displays the identification information in the display means, and further includes notifying means for performing vibration for notifying the medical professional of the location of the mobile communication terminal.

According to the above configuration, when the mobile communication terminal such as a smartphone, upon receiving the activation sign, does not return the identification information to an information processing device, and displays the identification information in the display means, a medical professional needs to locate the mobile communication terminal and confirm its display screen. In view of this, notifying means is provided, and the notifying means performs vibration upon receiving the activation sign, as a result, the location of the mobile communication terminal can be easily located.

Moreover, in the individual health information system of the present invention, the server transmits health information that is newly input, through wide area wireless communication network, on the medical institution side due to the examinee seeking consultation such as tests, treatment, procedures, and medication, to the mobile communication terminal through the wide area wireless communication network, and the health information recorded in the storage means is updated.

According to the above configuration, inputting of health information such as tests, treatment, procedures, and medication to a mobile communication terminal such as a smartphone is a difficult task for the examinee. Specifically, it is not realistic for a diagnostic image and the like to be shot by a built-in camera of a smartphone and imported to the smartphone. In view of this, on the medical institution side, every time health information that is used as an electronic health record or the like is newly input to the server and the health information is updated, the server transmits the health information to the mobile communication terminal of the examinee through a wide area wireless communication network such as a mobile phone channel, and the health information in the storage means is similarly updated.

Therefore, the examinee can record the latest health information in the mobile communication terminal without performing troublesome input work.

Also, in the individual health information system of the present invention, the health information recorded in the storage means of the mobile communication terminal is level-classified in advance, and there are cases where the level viewable by members on the mobile communication terminal is different from the level viewable by the medical professional as a result of performing a predetermined operation on the information processing device.

According to the above configuration, as a result of performing a predetermined operation such as canceling a password lock, the medical professionals such as paramedics and doctors can handle all health information so as to not hamper the emergency life-saving activities, and in contrast, there are cases where, on the member (examinee) side, even regarding information recorded in his/her mobile communication terminal, provision of the health information is limited.

Therefore, the health information can be more appropriately treated such as the information that a doctor or a family member has determined not to disclose is hidden from a specific member (examinee).

### INDUSTRIAL APPLICABILITY

An individual health information system according to the present invention provides an individual health information aggregation system for aggregating and managing health information such as medical records and test results of examinees spanning a plurality of medical institutions in an aggregation server of a management center in association with identification information (patient number) unique to each examinee, and enabling treatment and administration of medication to be appropriately carried out as a result of the examinee or a family member or a person connected with a medical institution viewing the heath information as appropriate, in which, in an accommodation place such as the inside of an ambulance, when the examinee is in poor physical condition such as when transported in an emergency situation, the system downloads and displays the health information on a screen as a result of a medical professional obtaining the identification information (patient number), and transmitting the identification information to the management center together with his or her own ID and password. It thus becomes possible for the health information of individuals that is aggregated and managed to also be used in emergencies, enabling the survival rate to be improved, and being suitable for emergency care.

1. An individual health information system, comprising:
a mobile communication terminal, wherein the mobile communication terminal is for recording health information of one or more members associated with an owner of the mobile communication terminal, the members comprising one or more of the owner and one or more individuals related to the owner of the mobile communication terminal, the mobile communication terminal comprising:
storage means for recording the health information;
receiving means for receiving a predetermined activation sign from outside;
a first communication interface for performing short range wireless communication with an information processing device; and
information control means for, upon the receiving means receiving the predetermined activation sign, transmitting the health information stored in the storage means from the first communication interface to the information processing device; and
the information processing device possessed by a medical professional that performs treatment when any of the members are in poor physical condition, the information processing device comprising:
a second communication interface; and
display means for, upon the second communication interface receiving the health information, displaying the health information.

2. The individual health information system according to claim 1, wherein the activation sign comprises one of calling by a predetermined voice of the medical professional or an emission of a predetermined tone of a transmitter.

3. The individual health information system according to claim 1, wherein the activation sign comprises an activation signal generated by activation signal generating means provided in the information processing device being transmitted from the second communication interface by the short range wireless communication, and received by the first communication interface of the mobile communication terminal.

4. The individual health information system according to claim 1,
wherein the first and second communication interfaces can perform bidirectional communication, and the information processing device further comprises selecting means of the health information, and
wherein the information processing device, when receiving of the health information from the mobile communication terminal becomes possible, transmits a result of a selection by the selecting means to the information control means from the second communication interface via the first communication interface, and the information control means reads out the health information that is the selection result from the storage means and transmits the health information to the information processing device.

5. The individual health information system according to according to claim 1, the mobile communication terminal further comprising:
input means through which the health information is input into the mobile communication terminal when transmission of health information to the information processing device becomes possible, wherein the information control means records the input health information to the storage means, and reads out the health information in response to an operation made on the input means and causes the display means to display the health information.

6. The individual health information system according to claim 1, further comprising:
a server that aggregates and holds aggregated health information of each of a plurality of examinees, each of the examinees being one of the members, that is collected in a plurality of medical institutions in association with unique identification information assigned in advance to that examinee, the aggregated health information comprising the health information and enables viewing of the aggregated health information of that examinee in the mobile communication terminal and information processing device based on a predetermined rule, in response to a request of at least one of the medical professional and the examinee;
a wide area wireless communication network to which the mobile communication terminal and the information processing device are connected,
wherein when the receiving means receives the predetermined activation sign from outside, the information control means displays unique identification information assigned in advance to the examinee on the display means, and
wherein the information processing device acquires the aggregated health information by accessing the server with the unique identification information assigned in advance to the examinee through the wide area wireless communication network.

7. The individual health information system according to claim 1 wherein a place where the member is accommodated when in poor physical condition is the inside one of an ambulance and an ambulance helicopter, and the medical professional that performs the treatment is at least one of a paramedic and a doctor.

8. The individual health information system according to claim 6, wherein the mobile communication terminal, when the receiving means receives the activation sign, displays the identification information on the display means, and further comprises notifying means for performing vibration for notifying the medical professional of the location of the mobile communication terminal.

9. The individual health information system according to claim 6, wherein the server, upon receiving the health information from one of the medical institutions upon that examinee seeking consultation from that medical institution, transmits the received health information, through the wide area wireless communication network, to the mobile communication terminal and causes the health information recorded in the storage means to be updated with the transmitted health information.

10. The individual health information system according to claim 1 wherein the health information recorded in the storage means of the mobile communication terminal is classified by levels in advance, and at least one of the levels viewable by members on the mobile communication terminal is different from the levels viewable by the medical professional as a result of performing a predetermined operation on the information processing device.

11. The individual health information system according to claim 2,
wherein the first and second communication interfaces can perform bidirectional communication, and the information processing device further comprises selecting means of the health information, and
wherein the information processing device, when receiving of the health information from the mobile communication terminal becomes possible, transmits a result of a selection by the selecting means to the information control means from the second communication interface via the first communication interface, and the information control means reads out the health information that is the selection result from the storage means and transmits the health information to the information processing device.

12. The individual health information system according to claim 2, the mobile communication terminal further comprising:
input means through which the health information is input into the mobile communication terminal when transmission of health information to the information processing device becomes possible, wherein the information control means records the received health information to the storage means, and reads out the health information in response to an operation made on the input means and causes the display means to display the health information.

13. The individual health information system according to claim 2, further comprising:
a server that aggregates and holds aggregated health information of each of a plurality of examinees, each of the examinees being one of the members, that is collected in a plurality of medical institutions in association with unique identification information assigned in advance to that examinee, the aggregated health information comprising the health information and enables viewing of the aggregated health information of that examinee in the mobile communication terminal and information processing device based on a predetermined rule, in response to a request of at least one of the medical professional and the examinee;
a wide area wireless communication network to which the mobile communication terminal and the information processing device are connected;
wherein when the receiving means receives the predetermined activation sign from outside, the information control means displays unique identification information assigned in advance to the examinee on the display means, and
wherein the information processing device acquires the aggregated health information by accessing the server with the unique identification information assigned in advance to the examinee through the wide area wireless communication network.

14. The individual health information system according to claim 13, wherein the mobile communication terminal, when the receiving means receives the activation sign, displays the identification information on the display means, and further comprises notifying means for performing vibration for notifying the medical professional of the location of the mobile communication terminal.

15. The individual health information system according to claim 13, wherein the server, upon receiving the health information from one of the medical institutions upon that examinee seeking consultation from that medical institution, transmits the received health information, through the wide area wireless communication network, to the mobile communication terminal and causes the health information recorded in the storage means to be updated with the transmitted health information.

16. The individual health information system according to claim 2, wherein a place where the member is accommodated when in poor physical condition is the inside one of an ambulance and an ambulance helicopter, and the medical professional that performs the treatment is at least one of a paramedic and a doctor.

17. The individual health information system according to claim 2, wherein the health information recorded in the storage means of the mobile communication terminal is classified by levels in advance, and at least one of the levels viewable by members on the mobile communication terminal is different from the levels viewable by the medical professional as a result of performing a predetermined operation on the information processing device.

18. The individual health information system according to claim 1, the mobile communication terminal further comprising:
a display unit on which a plurality of icons are displayed, wherein the display unit displays identifying information associated with one of the members upon one of the icons being selected, the identifying information comprising one or more of name, birth date, blood type, address, telephone number, and contact.

19. The individual health information system according to claim 18,
wherein upon the receiving means receiving the predetermined activation sign from outside, the display unit displays the identifying information and the mobile communication terminal provides notification regarding the mobile communication terminal's location.

20. The individual health information system according to claim 19, wherein the notification comprises one of sound, vibration, and light.
